(19) United States
(12) Patent Application Publication
(10) Pub. No.: US 2008/0269520 A1
(43) Pub. Date: Oct. 30, 2008

(54) Process for the Carbonylation of a Conjugated Diene to a Dicarboxylic Acid

(76) Inventors: Eit Drent, Amsterdam (NL); Rene Ernst, Amsterdam (NL); Willem Wabe Jager, Amsterdam (NL); Cornelia Alida Krom, Amsterdam (NL); Timothy Michael Nisbet, Amsterdam (NL)

Correspondence Address: SHELL OIL COMPANY, P O BOX 2463, HOUSTON, TX, 772522463, US

(21) Appl. No.: 11/884,103

(22) PCT Filed: Feb. 10, 2006
(86) PCT No.: PCT/EP2006/050829

§ 371 Date: May 13, 2008

(30) Foreign Application Priority Data
Feb. 11, 2005 (EP) 05101028.8

(51) Int. Cl. C07C 51/14

(52) U.S. Cl. 562/522

(57) ABSTRACT

A process for the carbonylation of a conjugated diene to a dicarboxylic acid, comprising the steps of (a) contacting a conjugated diene with carbon monoxide and water in the presence of a catalyst system including a source of palladium, a source of an anion and a bidentate phosphine ligand, to obtain a mixture comprising an ethylenically unsaturated acid and reversible diene adducts; (b) separating the obtained reaction mixture into a gaseous stream comprising unreacted conjugated diene and carbon monoxide, a first normally liquid stream comprising at least part of the ethylenically unsaturated acid and the reversible diene adducts, and a second normally liquid stream comprising the catalyst system in admixture with the ethylenically unsaturated acid; (c) recycling the second liquid stream obtained in step (b) to step (a); (d) separating the first liquid product stream obtained in step (b) into a stream comprising the ethylenically unsaturated acid and a stream comprising the reversible diene adducts; and (e) contacting the stream comprising the ethylenically unsaturated acid obtained in step (d) with carbon monoxide and water in the presence of a second catalyst system including a source of palladium, a source of an anion and a bidentate phosphine ligand.

### FIELD OF THE INVENTION

The present invention provides a process for the carbonylation of a conjugated diene to obtain an ethylenically unsaturated acid, and the subsequent carbonylation of the ethylenically unsaturated acid to a dicarboxylic acid.

### BACKGROUND OF THE INVENTION

Carbonylation reactions of conjugated dienes are well known in the art. In this specification, the term carbonylation refers to a reaction of a conjugated diene under catalysis by a transition metal complex in the presence of carbon monoxide and water, as for instance described in WO 04/103948.

In WO 04/103948, a process is disclosed for the preparation of adipic acid from 1,3-butadiene or a mixture of 1,3-butadiene with olefinic products in a two-stage reaction. In the first stage of the disclosed process, 1,3-butadiene was reacted with carbon monoxide and water in the presence of a carbonylation catalyst comprising a palladium compound, a source of an anion and 1,2-bis(di-tert-butylphosphinomethyl)benzene as bidentate diphosphine ligand for several hours until substantially all of the 1,3-butadiene was converted. In a second carbonylation step, additional water and carbon monoxide were added to the mixture comprising the catalyst and a mixture of 2-, 3- and 4-pentenoic acids obtained in the first carbonylation step, and the reaction was continued until at least part of the pentenoic acid product was converted to adipic acid.

Applicants have found that a specific Diels-Alder by-product is formed from at least one ethylenically unsaturated acid and the conjugated diene in the above process in significant amounts, which reduces the overall yield and purity of the desired product. It has now been found that formation of the by-product can be reduced.

### SUMMARY OF THE INVENTION

Accordingly, the subject invention provides a process for the carbonylation of a conjugated diene to a dicarboxylic acid, comprising the steps of

(a) contacting a conjugated diene with carbon monoxide and water in the presence of a catalyst system including a source of palladium, a source of an anion and a bidentate phosphine ligand, to obtain a mixture comprising an ethylenically unsaturated acid and reversible diene adducts formed by the conjugated diene with the ethylenically unsaturated acid;
(b) separating the obtained reaction mixture into a gaseous stream comprising unreacted conjugated diene and carbon monoxide, a first normally liquid product stream comprising at least part of the ethylenically unsaturated acid and the reversible diene adducts, and a second normally liquid stream comprising the catalyst system in admixture with the ethylenically unsaturated acid;
(c) recycling at least part of the second normally liquid stream obtained in step (b) to step (a);
(d) separating the first normally liquid product stream obtained in step (b) into a stream comprising the ethylenically unsaturated-acid and a stream comprising the reversible diene adducts; and
(e) contacting the stream comprising the ethylenically unsaturated acid product obtained in step (d) with carbon monoxide and water in the presence of a catalyst system including a source of palladium, a source of an anion and a bidentate phosphine ligand.

### FIGURES

FIG. 1 is a schematic representation of a preferred embodiment of the process according to the invention.

### DETAILED DESCRIPTION OF THE INVENTION

As illustrated in WO 04/103948, the product mixture obtained in the two-step process of WO 04/103948 comprises a particular by-product, i.e. 2-ethyl cyclohexene carboxylic acid (further referred to as ECCA), which is the Diels-Alder adduct of 1,3-butadiene and 2-pentenoic acid. Analogues of this by-product are expected to be formed from other conjugated dienes and their respective carbonylation products, i.e. ethylenically unsaturated acids. It was further found that if the catalyst is recycled from the further carbonylation of the ethylenically unsaturated acid to a dicarboxylic acid back to the first reaction step in admixture with the ethylenically unsaturated acid formed in the process, Diels-Alder products of the conjugated diene and the ethylenically unsaturated acid are formed in increasing amounts.

Without wishing to be bound to any particular theory, it is believed that in the case of carbonylation of for instance pentenoic acid, the specific by-products are formed due to the fact that the carbonylation catalyst isomerises the 3-pentenoic acid or 4-pentenoic acid initially formed to 2-pentenoic acid. The 2-pentenoic acid then reacts with 1,3-butadiene under formation of ECCA. In this thermal Diels-Alder reaction, the ethylenically unsaturated double bound of the 2-pentenoic acid acts reacts as dienophile with the conjugated diene double bonds under formation of a substituted cyclohexene ring. The formation of ECCA has already been illustrated in the reaction disclosed in WO 04/103948.

Although this by-product formation has only been described for the carbonylation of 1,3-butadiene to adipic acid as a saturated dicarboxylic acid, it is assumed that other conjugated dienes will form similar Diels-Alder adducts that with their respective ethylenically unsaturated acid products. The present process permits to minimise the residence time of the initially formed ethylenically unsaturated acid mixture in presence of the catalyst system, by removing the ethylenically unsaturated acid from the reaction mixture comprising the catalyst system.

Within the context of this specification, the terms “dicarboxylic acid” and “ethylenically unsaturated acid” may each describe a single compound or a mixture of isomers, depending on the structure of the conjugated diene employed. In case of 1,3-butadiene as conjugated diene, the term “ethylenically unsaturated acid” describes 2-pentenoic acid, 3-pentenoic acid and 4-pentenoic acid, and mixtures thereof. The dicarboxylic acid may preferably be a saturated diacid, since both double bonds of the conjugated diene are converted, although other unsaturated bonds in the conjugated diene may not be affected, for instance in case of a further alkyne- or cyano-functionalized conjugated diene.

It was found that in step (a) of the subject process, conjugated dienes have the tendency to reversibly form esters with any carboxylic acid present in the reaction mixture, in particular under catalysis by the carbonylation catalyst.

Depending on the reaction conditions and on the nature of the conjugated diene, such alkenyl esters can be formed in substantial amounts. Without wishing to be bound to any particular theory, it is believed that the formation of the esters from the conjugated diene and carboxylic acids present in the reaction mixture, such as the ethylenically unsaturated acid product formed, is an equilibrium reaction catalyzed by the carbonylation catalyst, albeit at a comparatively slow rate. The presence of a high diene concentration, as well as an increasing amount of ethylenically unsaturated acid favours the formation of esters. In absence of catalyst, the equilibrium reaction becomes very slow, hence effectively freezing the equilibrium.

Since the alkenyl esters can be reverted into the conjugated diene and the carboxylic acid, such as the ethylenically unsaturated acid formed, they are referred to as “reversible diene adducts” throughout the present specification. These “reversible diene adducts” were found to be remarkably stable in absence of the carbonylation catalyst. In the case of 1,3-butadiene as conjugated diene, the “reversible diene adducts” are butenyl esters with any carboxylic acid present in the reaction mixture, thus mainly butenyl esters of 2-, 3- and 4-pentenoic acid, and mixtures thereof.

The Diels-Alder by-product, ECCA in the case of 1,3-butadiene as conjugated diene, is not reversible under the conditions of the carbonylation, since a retro- the Diels-Alder reaction requires a substantially higher amount of energy, and therefore not considered a reversible diene adduct within the subject specification. The subject process has the object of reducing its formation.

In step (a), the conjugated diene is contacted with carbon monoxide and water in the presence of a catalyst system including a source of palladium, a source of an anion and a bidentate phosphine ligand, to obtain a mixture comprising an ethylenically unsaturated acid product and reversible diene adducts. Then the conjugated diene and reversible diene adducts are removed from the reaction mixture in step (b). Step (a) of the present process is not allowed to proceed to full conversion of the conjugated diene and its reversible adducts, but only to partial conversion.

In particular in the case of the carbonylation of 1,3-butadiene, step (a) is preferably allowed to proceed to no more than 99% of conversion, based on moles of 1,3-butadiene converted versus moles of 1,3-butadiene fed. Yet more preferably, step (a) is allowed to proceed to 85% of conversion, again more preferably to 75% of conversion, again more preferably step to 65% of conversion, and most preferably step (a) is allowed to proceed to 30 to 60% of conversion, based on moles of 1,3-butadiene converted versus moles of 1,3-butadiene fed.

In step (a), the ratio (v/v) of diene and water in the feed can vary between wide limits and suitably lies in the range of 1:0.0001 to 1:500. However, it was found that the addition of water in step (a) to the reaction medium in order to provide a higher concentration of the reactant and hence an increased reaction rate had the opposite effect, i.e. an increase of the water concentration resulted in a strongly decreased reaction rate. Therefore, preferably, in step (a), less than 3% by weight of water is present in the reactor, yet more preferably, less than 2% by weight of water, yet more preferably, less than 1% by weight of water, again more preferably less than 0.15% by weight of water, and most preferably from 0.001% to less than 3% by weight of water (w/w) is present in the reactor, calculated on the total weight of liquid reaction medium. Again more preferably, these water concentrations are maintained continuously at this level, in particular if the reaction is performed as semi-batch or as continuous process. The water concentration may be determined by any suitable method, for instance by a Karl-Fischer-titration.

In step (b), the reaction mixture obtained in step (a) is separated into a gaseous stream comprising unreacted conjugated diene and carbon monoxide, a first normally liquid product stream comprising at least part of the ethylenically unsaturated acid and the reversible diene adducts, and a second normally liquid stream comprising the catalyst system in admixture with the ethylenically unsaturated acid. “Normally liquid” within the context of the present specification has the meaning that a stream is a liquid under normal conditions, i.e. normal pressure and normal temperature.

Step (b) may be performed by any known suitable separation method.

Since the reaction in step (a) is performed under carbon monoxide pressure, release of the pressure allows removing unreacted carbon monoxide together with normally gaseous conjugated diene. The conditions for the removal of the gaseous stream may conveniently be chosen such that the conjugated diene is gaseous at the separation conditions, for instance by adjusting pressure and temperature. Preferably, step (b) is performed as a distillative separation, more preferably a flash separation under reduced pressure. If 1,3-butadiene is the conjugated diene, the flash separation is preferably performed at a bottom temperature in range of from 70 to 150° C. and a pressure of from 1 to 30 kPa (10 to 300 mbar), yet more preferably at a bottom temperature in range of from 90 to 130° C. and a pressure of from 2.5 to 15 kPa, and most preferably, at a bottom temperature in the range of from 100 to 110° C. and at a pressure in the range of from 3 to 8 kPa. Although these pressures and temperatures are not critical, pressures of above 20 kPa should be avoided due to the high temperatures required, which may result in catalyst degradation, while pressures below 1 kPa will require specific equipment. Preferably, the flash separation is performed in a film evaporator, more preferably in a falling film or wiped film evaporator, since these allow high throughput and short catalyst residence time.

As a result, a gaseous stream, and a first normally liquid product stream and a second normally liquid product stream are obtained in step (b).

The first normally liquid product stream comprises part of the ethylenically unsaturated acid formed in step (a), as well as the reversible diene adducts. The amount of ethylenically unsaturated acid in this stream is limited solely by the catalyst concentration remaining in the second liquid stream, which is the bottom stream. If too much ethylenically unsaturated acid is removed from the bottom stream, then catalyst degradation may occur in the remaining concentrate, or catalyst components or side-products can crystallize and obstruct the recycling operation. Preferably, in a continuous process, at least 5% of the ethylenically unsaturated acid is comprised in the first liquid stream, while 95% remain in the bottom stream and hence are recycled. More preferably, the ratio of the ethylenically unsaturated acid in the first liquid (overhead) stream versus the second liquid (bottom) stream is in the range of from 30:70 to 90:10, again more preferably in the range of from 60:40 to 80:20. In this way, most ethylenically unsaturated acid is withdrawn from the reactor, and hence from the presence of the conjugated diene. As a result, the formation of by-products such as ECCA is reduced. Additionally, the catalyst is not exposed to higher temperatures for a prolonged period of time. This increases the catalyst stability, and thus allows higher turn over numbers.

In step (c), the second liquid stream comprising the catalyst system in admixture with ethylenically unsaturated acid product obtained in step (b) is recycled to step (a), subject to an optional catalyst purge while the gaseous stream is preferably recycled to step (a). In this purge, undesired side-products such as ECCA, or any conjugated diene oligomer or polymer may be advantageously be removed from the catalyst stream.

In step (d), the first liquid product stream obtained in step (b) is separated into a stream comprising the ethylenically unsaturated acid product and a stream comprising the reversible diene adducts. This is preferably done in a distillative separation. In the case of 1,3-butadiene, the reversible diene adducts and the pentenoic acid mixture have sufficiently different boiling ranges to allow a complete separation in a simple distillation column.

The obtained mixture comprising the reversible diene adducts, usually also comprising some ethylenically unsaturated acid and other by-products, is then either directly recycled to step (a), or converted in a separate conversion step in the presence of a suitable catalyst back into conjugated diene and ethylenically unsaturated compound. At this point in the process, any undesired side-products, such as non-functionalized Diels-Alder products of the conjugated diene, for instance 4-vinyl cyclohexene (4-VCH) may conveniently be removed from the first normally liquid stream.

For the conversion, the reversible diene adducts are preferably contacted with a suitable catalyst before recycling the obtained conjugated diene and the unsaturated acid back to the process. Any catalyst suitable for the conversion may be applied, such as heterogeneous or homogeneous palladium catalysts. An example of a suitable palladium catalyst is the catalyst system as described for step (a). The obtained conjugated diene is then preferably recycled to step (a), whereas the ethylenically unsaturated acid product may be recycled to step (a) or combined with the stream comprising the ethylenically unsaturated acid product obtained in step (d).

The first stream obtained in step (d) comprising the ethylenically unsaturated acid product is then subjected to a further carbonylation step (e) in the presence of a second carbonylation catalyst system, to obtain a mixture comprising the dicarboxylic acid product in admixture with the ethylenically unsaturated acid product.

To this end, the stream comprising the ethylenically unsaturated acid product obtained in step (d) is contacted with carbon monoxide and water in the presence of a second catalyst system including a source of palladium, a source of an anion and a bidentate phosphine ligand, to obtain a mixture comprising the saturated dicarboxylic acid in admixture with the ethylenically unsaturated acid product.

Since no conjugated diene is present in this second carbonylation step, not only does the reaction proceed smoothly and does not require long induction times, but also no ECCA can be formed. As a result, the isomerisation of the ethylenically unsaturated acid into the electronically most stable isomer is not critical in this reaction step.

It was found that in step (e) an increase of the water concentration resulted in a strongly increased reaction rate. Therefore, preferably, in step (e), the water concentration (w/w) on the reaction mixture is maintained within the range of from to 1 to 50% (w/w), preferably from 2 to 30% (w/w), more preferably from 3 to 25% (w/w), yet more preferably from 4 to 15% (w/w), and most preferably from 5 to 10% (w/w), calculated on the total weight of reactants.

The present process preferably comprises a further reaction step (f) of separating the dicarboxylic acid from a liquid stream comprising the ethylenically unsaturated acid and the second catalyst system. In the case of 1,3-butadiene, the dicarboxylic acid is isolated from the reaction mixture by crystallization of the dicarboxylic acid in the reaction mixture and separation of the dicarboxylic acid crystals from the remaining reaction mixture containing the catalyst. It has been found that the dicarboxylic acid crystals can be obtained in a high purity in a single or only few crystallization steps, making it an efficient method for the separation of the product from the catalyst and unreacted ethylenically unsaturated acid intermediate.

The remaining reaction mixture containing the catalyst system in admixture with ethylenically unsaturated acid is then preferably recycled to step (e). Although this catalyst stream could also be recycled to step (a), this is however avoided since the ethylenically unsaturated acid product is increasingly isomerized, which would result in an increase in the formation of products such as ECCA.

Operating two separate catalyst recycles over step (a) and (e) has the further advantage that the water concentration in each recycle does not have to be adapted to the preferred ranges for the carbonylation reactions.

Process steps (a) to (e) are preferably performed in a continuous operation. Steps (a) and (e) of the subject process are suitably performed in a cascade of reactors suitable for gas-liquid reactions, such as constant flow stirred tank reactor, or a bubble column type reactor, as for instance described in “Bubble Column Reactors” by Wolf-Dieter Deckwer, Wiley, 1992. A bubble column reactor is a mass transfer and reaction device in which in one or more gases are brought into contact and react with the liquid phase itself or with a components dissolved or suspended therein. Preferably, a reactor with forced circulation is employed, which are generally termed “ejector reactors”, or if the reaction medium is recycled to the reactor, “ejector loop reactors”. Such ejector reactors are for instance described in U.S. Pat. No. 5,159,092 and JP-A-11269110, which employ a liquid jet of the liquid reaction medium as a means of gas distribution and circulation.

The present process may optionally be carried out in the presence of a solvent, however preferably the acid serving as source of anions is used as the reaction solvent. Most preferably, though, the reaction is performed in the ethylenically unsaturated acid products and/or the dicarboxylic acid product, provided the mixture remains liquid at reaction conditions.

The subject process permits to react conjugated dienes with carbon monoxide and a co-reactant. The conjugated diene reactant has at least 4 carbon atoms. Preferably the diene has from 4 to 20 and more preferably from 4 to 14 carbon atoms. However, in a different preferred embodiment, the process may also be applied to molecules that contain conjugated double bonds within their molecular structure, for instance within the chain of a polymer such as a synthetic rubber. The conjugated diene can be substituted or non-substituted. Preferably the conjugated diene is a non-substituted diene. Examples of useful conjugated dienes are 1,3-butadiene, conjugated pentadienes, conjugated hexadienes, cyclopentadiene and cyclohexadiene, all of which may be substituted. Of particular commercial interest are 1,3-butadiene and 2-methyl-1,3-butadiene (isoprene). Examples of suitable catalyst systems as described above are those disclosed in EP-A-1282629, EP-A-1163202, WO2004/103948 and/or WO2004/103942. In the subject process, the first and the second catalyst are preferably identical, although the two possible catalyst recycling streams are not combined, i.e. preferably no catalyst is recycled from step (e) to step (a).

Suitable sources of palladium for steps (a) and (e) include palladium metal and complexes and compounds thereof such as palladium salts; and palladium complexes, e.g. with carbon monoxide or acetyl acetonate, or palladium combined with a solid material such as an ion exchanger. Preferably, a salt of palladium and a carboxylic acid is used, suitably a carboxylic acid with up to 12 carbon atoms, such as salts of acetic acid, propionic acid and butanoic acid. A very suitable source is palladium (II) acetate.

Any bidentate diphosphine that could form an active carbonylation catalyst with palladium may be used in the subject process. Preferably, a bidentate diphosphine ligand of formula R¹R²P—R—PR³R⁴ is employed, in which ligand R represents a divalent organic bridging group, and R¹, R², R³ and R⁴ each represent an organic group that is connected to the phosphorus atom through a tertiary carbon atom due to the higher activity found with such catalysts in both reaction steps. Yet more preferably, R represents an aromatic bidentate bridging group that is substituted by one or more alkylene groups, and wherein the phosphino groups R¹R²P— and —PR³R⁴ are bound to the aromatic group or to the alkylene group due to the observed high stability of these ligands. Most preferably R¹, R², R³ and R⁴ are chosen in such way, that the phosphino group PR¹R² differs from the phosphino group PR³R⁴.

The ratio of moles of a bidentate diphosphine per mole atom of palladium is not critical. Preferably it ranges from 0.5 to 100, more preferably from 0.9 to 10, yet more preferably from 0.95 to 5, yet more preferably in the range of 3 to 1, again more preferably in the range of 2 to 1. In the presence of oxygen, slightly higher than stoichiometric amounts are beneficial. The source of anions preferably is an acid, more preferably a carboxylic acid, which can serve both as catalyst component, as well as solvent for the reaction.

Again more preferably, the source of anions is an acid having a pKa above 2.0 (measured in aqueous solution at 18° C.), and yet more preferably an acid having a pKa above 3.0, and yet more preferably a pKa of above 3.6.

Examples of preferred acids include carboxylic acids, such as acetic acid, propionic acid, butyric acid, pentanoic acid, pentenoic acid and nonanoic acid, the latter three being highly preferred as their low polarity and high pKa was found to increase the reactivity of the catalyst system.

The molar ratio of the source of anions, and palladium is not critical. However, it suitably is between 2:1 and 10⁷:1 and more preferably between 10²:1 and 10⁶:1, yet more preferably between 10²:1 and 10⁵:1, and most preferably between 10²:1 and 10⁴:1 due to the enhanced activity of the catalyst system. Very conveniently the acid corresponding to the desired product of the reaction can be used as the source of anions in the catalyst. 2-, 3- and/or 4-pentenoic acid is particularly preferred in case the conjugated diene is 1,3-butadiene. Preferably the reaction is conducted in pentenoic acid, since this was found to not only form a highly active catalyst system, but also was found to be a good solvent for all reaction components. Moreover, the high boiling point of these compounds allows performing step (b) without the need for a separation of components, and also allows maintaining the catalyst in solution for recycling from step (c) to step (a). The quantity in which the complete catalyst system is used usually amounts in the range of 10⁻⁸ to 10⁻¹, preferably in the range of 10⁻⁷ to 10⁻² mole atom of palladium per mole of conjugated diene are used, preferably in the range of 10⁻⁵ to 10⁻² mole atom per mole. However, in the case of 1,3-butadiene, it was found that if the amount of catalyst is chosen at a level below 20 ppm, calculated on the total amount of liquid reaction medium, Diels-Alder reactions of the conjugated diene will become more prominent. In the case of 1,3-butadiene, these side-products include other than ECCA also 4-vinyl cyclohexene (further referred to as VCH, being the adduct of two 1,3-butadiene molecules). Accordingly, in step (a), the carbonylation is preferably performed in the presence of at least 20 ppm of catalyst, more preferably in the presence of 100 ppm of catalyst, and most preferably in the presence of at least 500 ppm.

The carbonylation reaction according to the present invention in steps (a) and (e) is carried out at moderate temperatures and pressures. Suitable reaction temperatures are in the range of 0-250° C., more preferably in the range of 50-200° C., yet more preferably in the range of from 80-150° C.

The reaction pressure is usually at least atmospheric pressure. Suitable pressures are in the range of 0.1 to 25 MPa (1 to 250 bar), preferably in the range of 0.5 to 15 MPa (5 to 150 bar), again more preferably in the range of 1 to 9.5 MPa (5 to 95 bar) since this allows use of standard equipment. Carbon monoxide partial pressures in the range of 0.1 to 9 MPa (1 to 90 bar) are preferred, the upper range of 5 to 9 MPa being more preferred. Higher pressures require special equipment provisions, although the reaction would be faster since it was found to be first order with carbon monoxide pressure.

Carbon monoxide can be used in its pure form or diluted with an inert gas such as nitrogen, carbon dioxide or noble gases such as argon, or co-reactant gases such as ammonia. The subject process further preferably comprises a further process step (g) of separating and optionally purifying the dicarboxylic acid obtained in step (e). The process further preferably comprises the steps of (i) converting the dicarboxylic acid to its dichloride, and (ii) reacting the dicarboxylic acid dichloride with a diamine compound to obtain an alternating co-oligomer or co-polymer.

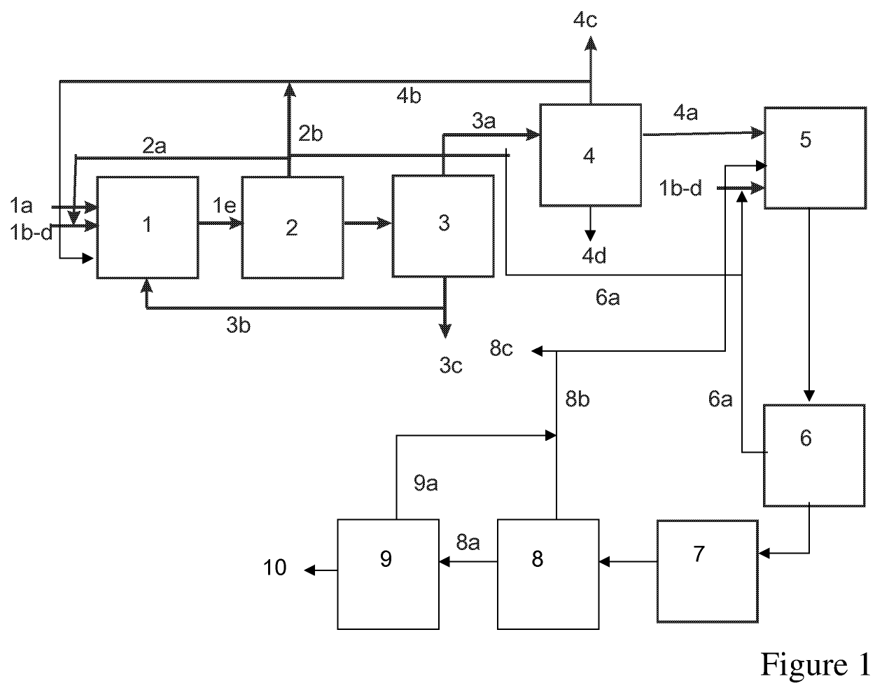

The invention will further be described by way of example with reference to FIG. 1. FIG. 1 is a schematic representation of a preferred embodiment of the process according to the present invention. FIG. 1 illustrates a process wherein a conjugated diene (1a), carbon monoxide (1b), water (1c) and a catalyst system including a source of palladium, a source of an anion and a bidentate phosphine ligand (1d) are supplied to a reactor (1). In this reactor (1), the conjugated diene is contacted with the carbon monoxide and water in the presence of a catalyst system including a source of palladium, a source of an anion and a bidentate phosphine ligand, to obtain a mixture (1e) comprising an ethylenically unsaturated acid. The mixture (1e) is then depressurized (2) to obtain a depressurized mixture. At this stage, optionally a stream of unreacted carbon monoxide (2a) and a stream of a normally gaseous conjugated diene (2b) and may be separated from the mixture, and recycled to reactor (1). The depressurized mixture is then transferred to a flash vessel (3), wherein a stream (3a) comprising the remaining conjugated diene, reversible diene adducts and part of the ethylenically unsaturated acid product is separated from a bottom stream (3b) comprising the catalyst system in admixture with part of the ethylenically unsaturated acid. The catalyst stream (3b) is then in full recycled to reactor (1), or in part, subject to an optional catalyst purge (3c). The stream (3a) comprising the remaining conjugated diene, reversible diene adducts and part of the ethylenically unsaturated acid is subjected to a distillation (9), wherein a stream (9b) comprising the reversible diene adducts in admixture with part of the ethylenically unsaturated acid, a second stream (4a) comprising a major portion of the ethylenically unsaturated acid, and a bottom stream (4d) comprising Diels-Alder products of the conjugated diene and the ethylenically unsaturated acid are separated from each other. A stream (4b) comprising the conjugated diene and carbon monoxide is recycled to the reactor (1), after optional removal of Diels-Alder products of the conjugated diene (4c). The remaining mixture (4a) comprising mainly ethylenically unsaturated acid is transferred to a reactor (5), where it is reacted further under carbon monoxide pressure (1b) with additional water (1c) and a catalyst system including a source of palladium, a source of an anion and a bidentate phosphine ligand (1d) to obtain a stream comprising the dicarboxylic acid in admixture with the ethylenically unsaturated acid and the catalyst system. The obtained mixture is then depressurized (6), while remaining carbon monoxide (6a) is recycled to step (1) or step (5). The depressurized mixture is then cooled (7), and subjected to filtration of the obtained dicarboxylic acid (8), yielding crude dicarboxylic acid (8a) and a liquid filtrate (8b). The liquid filtrate (8b) comprising the catalyst system in admixture with the ethylenically unsaturated acid is recycled to step (5), subject to an optional purge (8c). The crude dicarboxylic acid (8a) may then be subjected to purification (9), yielding purified dicarboxylic acid (10). Catalyst and ethylenically unsaturated acid left in the crude dicarboxylic acid (8a) are thereby removed as stream (9a) and combined with stream (8b).

1. A process for the carbonylation of a conjugated diene to a dicarboxylic acid, comprising the steps of
(a) contacting a conjugated diene with carbon monoxide and water in the presence of a catalyst system including a source of palladium, a source of an anion and a bidentate phosphine ligand, to obtain a mixture comprising an ethylenically unsaturated acid and reversible diene adducts;
(b) separating the obtained reaction mixture into a gaseous stream comprising unreacted conjugated diene and carbon monoxide, a first normally liquid stream comprising at least part of the ethylenically unsaturated acid and the reversible diene adducts, and a second normally liquid stream comprising the catalyst system in admixture with the ethylenically unsaturated acid;
(c) recycling the second liquid stream obtained in step (b) to step (a);
(d) separating the first liquid product stream obtained in step (b) into a stream comprising the ethylenically unsaturated acid and a stream comprising the reversible diene adducts; and
(e) contacting the stream comprising the ethylenically unsaturated acid obtained in step (d) with carbon monoxide and water in the presence of a second catalyst system including a source of palladium, a source of an anion and a bidentate phosphine ligand.

2. The process of claim 1, further comprising a step (f) of separating the dicarboxylic acid obtained in step (e) from a liquid stream comprising the second catalyst system in admixture with the ethylenically unsaturated acid.

3. The process of claim 2, wherein the liquid stream obtained in step (f) comprising the ethylenically unsaturated acid and the second catalyst system is recycled to step (e).

4. The process of claim 1, wherein the reversible diene adducts are recycled to step (a).

5. The process of claim 1, wherein the reversible diene adducts are converted to the conjugated diene and the ethylenically unsaturated acid by contacting them with a suitable catalyst, and wherein the obtained the conjugated diene is recycled to step (a).

6. The process of claim 1, wherein the water concentration in step (a) is maintained at from 0.001 to less than 3% by weight of water, calculated on the overall weight of the liquid reaction medium.

7. The process of claim 1, wherein the water concentration in step (e) is maintained at a range of from 1 to 50% by weight of water, calculated on the overall weight of the liquid reaction medium.

8. The process of claim 1, wherein the ethylenically unsaturated acid is employed as solvent for the process.

9. The process of claim 1, wherein the conjugated diene is 1,3-butadiene.

10. The process of claim 1, wherein the bidentate diphosphine ligand of formula R¹R²P—R—PR³R⁴ is employed, in which ligand R represents a divalent organic bridging group, and R¹, R², R³ and R⁴ each represent an organic group that is connected to the phosphorus atom through a tertiary carbon atom.

11. The process of claim 1, wherein the separation of first normally liquid stream and the second normally liquid stream in step (b) is performed in a film evaporator.

12. The process claim 11, wherein the first normally liquid stream is separated as top product, and the second normally liquid stream as bottom product.

13. The process of claim 11, wherein the film evaporator is a falling film evaporator or wiped film evaporator.

14. The process of claim 1, further comprising a step (g) of purifying the dicarboxylic acid obtained in step (d).

15. The process of claim 2, further comprising the steps of
(i) converting the dicarboxylic acid to its dichloride, and
(ii) reacting the dicarboxylic acid dichloride with a diamine compound to obtain an alternating co-oligomer or co-polymer.
